(19) United States
(12) Patent Application Publication
(10) Pub. No.: US 2011/0046302 A1
(43) Pub. Date: Feb. 24, 2011

(54) Polymer Compositions

(75) Inventors: Joaquin Bigorra Llosas, Sabadell (ES); Ivo Reetz, Barcelona (ES); Javier Raya, Sant Vicenc dels Horts (ES); Teresa Mas, STA Coloma de Queralt (ES); Rainer Hofer, Dusseldorf (DE); Julio Puche Artal, Barcelona (ES)

Correspondence Address: FOX ROTHSCHILD LLP, 997 Lenox Drive, Bldg. #3, Lawrenceville, NJ, 08648, US

(73) Assignee: COGNIS IP MANAGEMENT GMBH, Dusseldorf (DE)

(21) Appl. No.: 12/446,920

(22) PCT Filed: Oct. 13, 2007
(86) PCT No.: PCT/EP2007/008911

§ 371 Date: Nov. 1, 2010

(30) Foreign Application Priority Data
Oct. 23, 2006 (EP) EP06022122.3

(51) Int. Cl. C08K 3/20; C09D 175/04; C07C 233/02

(52) U.S. Cl. 524/591; 524/871; 564/192

(57) ABSTRACT

Disclosed are polymer compositions, comprising (a) at least one polymer, and (b) at least one dialkylamide, with the exception of dialkylformamides and dialkylacetamides.

### CROSS-REFERENCE TO RELATED APPLICATIONS

This application is the National Phase entry of PCT/EP2007/008911, filed Oct. 13, 2007, which claims priority to EPO patent application number EP06022122.3 filed Oct. 23, 2006, both of which are incorporated herein by reference in their entireties.

### FIELD OF THE INVENTION

The present invention relates to polymer compositions, more particularly to polyurethane dispersions comprising environmentally friendly solvents.

### BACKGROUND OF THE INVENTION

Polymer compositions, in particular dispersions of polyurethanes in mixtures of water and organic solvents, are known, for example, for the final treatment of leather, textile, fibers, wood, and other coating applications. The aim of using these polymers is to create basic physical and chemical fastnesses, or to change the feel of the material. Usually, the polymer compositions comprise solvents, such as N-methylpyrrolidone (NMP), dimethylformamide (DMF), dimethylacetamide (DMA) or xylene, which are chemically inert and reduce viscosity during the composition preparation. Nevertheless, these organic solvents are considered to be hazardous to human health and not very environmentally friendly.

It has therefore been the object of the present invention to provide new polymer compositions, particularly new polyurethane dispersions comprising solvents which behave in the same manner as well known solvents such as NMP, DMF, DMA or xylene, but are less toxic and show better biodegradability.

### DETAILED DESCRIPTION OF PREFERRED EMBODIMENTS

The present invention claims polymer compositions, comprising
(a) at least one polymer and
(b) at least one dialkylamide, except for dialkylformamides and dialkylacetamides.

Surprisingly it has been found that dialkylamides, preferably those which are liquid at 20° C. and are based on acids having a chain length of at least 3 carbon atoms, preferably 6 to 22 carbon atoms, and more preferably 8 to 12 carbon atoms, exhibit the required properties.

Dialkylamides

According to the present invention it has been found that both dialkylamides based on mono and dicarboxylic acids are useful to act as solvents for the manufacture of the dispersions.

Therefore, in a first embodiment, suitable dialkylamines based on fatty acids follow the general formula (I),

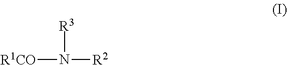

in which R¹CO stands for an aliphatic or aromatic acyl moiety having 6 to 22 carbon atoms, preferably 8 to 12 carbon atoms and 0 or 1 to 3 double bonds, and R² and R³ independently from each other represent a C₁-C₄ alkyl group. Typical examples are dialkylamides based on caproic acid, caprylic acid, 2-ethyl hexanoic acid, caprinic acid, lauric acid, myristic acid, palmitic acid, stearic acid, isostearic acid, oleic acid, (conjugated) linoleic acid, linolenic acid, gadoleic acid, arachidonic acid, behenic acid, erucic acid, tall oil fatty acid, and their technical mixtures or benzoic acid. Examples of suitable alkyl groups are methyl, ethyl, propyl, i-propyl, n-butyl, i-butyl, and tert.-butyl.

In a second embodiment, said dialkylamides can be derived from dicarboxylic acids and follow the general formula (II)

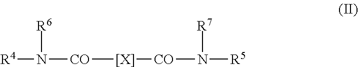

in which R⁴, R⁵, R⁶ and R⁷ independently from each other represent a C₁-C₄ alkyl or hydroxyalkyl group and X stands for an alkylene group having 1 to 12 carbon atoms. Typical examples are the symmetrical or asymmetrical diamides based on maleic acid, fumaric acid or adipic acid. Examples of suitable alkyl groups are again methyl, ethyl, propyl, i-propyl, n-butyl, i-butyl, and tert.-butyl.

In a first preferred embodiment of the present invention, those dialkylamides are used which show similar alkyl groups, preferably methyl groups, since dimethylamides exhibit superior solvent properties. Therefore, the residues R², R³, R⁴, R⁵, R⁶ and R⁷ in formulae (I) and (II) preferably represent methyl groups. The species showing the best solvent properties can be found in the group comprising the C₆-C₁₂ fatty acid dimethylamides, which are especially preferred for the purpose of the present invention.

The relationship between the polymers, in particular the polyurethane, and the dialkylamides is not critical, and depends on the solubility properties of the polymer, and on the technical application for which the products are desired. In particular for polyurethane dispersions, the composition typically comprises
(a) about 3 to about 50 , preferably about 5 to about 35% by weight (b.w.) of polymer; and
(b) about 1 to about 35, preferably about 5 to about 15% b.w. of dialkylamides

provided that the amounts add up to 100% b.w. with water.

### Manufacturing of the Polymer Compositions

A further object of the present invention is directed to a process for making polymer compositions, according to which one or more diols is treated with an isocyanate in the presence of a catalyst and a dialkylamide solvent to obtain the polyurethane. When a diol comprises free carboxylic acid groups, the resulting polyurethane is preferably neutralised with trialkylamines, for example triethanolamine, in order to obtain a water-soluble or water-dispersible anionic final product. The compositions may be solutions or dispersions.

Typical examples of suitable polymers according to the present invention are polyolefins, polyesters, polyamides, and preferably, polyurethanes. Selection of the monomers is driven by the desired application for the polymer obtained from the process. In case of the preferred isocyanates and with respect to an application for treatment of leather and textiles isophorone diisocyanate (IPDI), toluene diisocyanate or methylene bis-(4-cyclohexylisocyanate) (H₁₂MDI) and a polyether diol with varying chain length, a polyester diol or a polycarbonate diol have been found advantageous.

### INDUSTRIAL APPLICATION

As explained above, the dialkylamides show very good solvent properties during the manufacture of the polymer dispersions and do not cause any negative effects during the application of the dispersions, for example in the course of leather, fibre or textile finishing.

Further objects of the present invention are directed to the use of dialkylamides as solvents for the manufacture of polymer compositions and the use of said polymer compositions for the treatment of leather, fibres and/or textiles, and the coating of surfaces.

### EXAMPLES

Example 1

### Manufacture of a Leather Auxiliary Agent

20.9 kg of polytetrahydrofuran (PTHF 1000) together with 2 kg of dimethylolpropionic acid and 11.3 kg of a technical grade C₈-C₁₀ fatty acid dimethylamide were placed into a 100-L reactor. The mixture was heated to 50° C. with stirring for 30 min, until homogeneous. At 50° C., a first portion (50%) of a total of 7.7 kg of isophorone diisocyanate (IPDI) and 4 g of dibutyltin laurate catalyst were added. The reaction temperature was increased to 95° C., where it was maintained for 1 hour. The second portion of the IPDI was then added and the reaction continued at 95° C. for a further hour. The temperature was then lowered to 70° C. and the isocyanate number (NCO) was determined. Once the theoretical NCO value was achieved, the carboxylic groups were neutralized with triethanol amine (TEA). The temperature was maintained at 70° C. for 30 min. A dispersion was obtained by slowly adding the prepolymer into a second reactor containing 64 kg of water and 1.2 kg of hydrazine monohydrate chain extender, while maintaining the temperature at or below 40° C. The resulting low viscosity polyurethane dispersion was useful for the finishing of leather.

1. A Polymer composition, comprising;
(a) at least one polymer; and
(b) at least one dialkylamide, with the exception of dialkylformamides and dialkylacetamides.

2. The composition of claim 1, wherein said polymer is a polyurethane.

3. The composition of claim 1, wherein said dialkylamide follows the general formula (I), 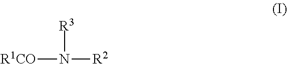
wherein R¹CO represents an aliphatic or aromatic acyl moiety having 6 to 22 carbon atoms and 0 or 1 to 3 double bonds, and R² and R³ independently from each other represent a C₁-C₄ alkyl radical group.

4. The composition of claim 1, wherein said dialkylamide follows the general formula (II), 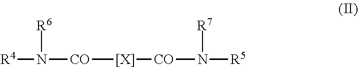
wherein R⁴, R⁵, R⁶ and R⁷ independently represent a C₁-C₄ alkyl radical group and X stands for represents an alkylene group having 1 to 12 carbon atoms.

5. The composition of claim 3, that wherein R² and R³ are methyl.

6. The composition of claim 1, wherein said dialkylamide is a C₆-C₁₂ fatty acid dimethylamide.

7. The composition of claim 1, comprising:
(a) about 3 to about 50% b.w. of polymers; and
(b) about 1 to about 35% b.w. of dialkylamides
provided that the amounts add up to 100% b.w. with water.

8. The composition of claim 1, wherein said composition is either a solution or dispersion.

9. A process for making aqueous polymer compositions, comprising treating one or more diols with an isocyanate in the presence of a catalyst and a dialkylamide as solvent to obtain a polymer dispersion or solution.

10. (canceled)

11. (canceled)

12. The composition of claim 4, wherein R⁴, R⁵, R⁶ and R⁷ are methyl.

13. A method of manufacturing aqueous polymer compositions, comprising using solvents comprising dialkylamides, excluding dialkylformamides and dialkylacetamides.

14. A method for the treatment of leather, fibers and/or textiles, and the coating of surfaces, comprising contacting said leather, fibers, textiles or surfaces with the aqueous polymer composition of claim 1.
